(19) United States
(12) Patent Application Publication
(10) Pub. No.: US 2009/0263356 A1
(43) Pub. Date: Oct. 22, 2009

(54) ANTI-ANGIOGENIC COMPOSITION COMPRISING GRAIN-DERIVED COMPONENT AS ACTIVE INGREDIENT

(75) Inventors: Toshiro Omori, Oita (JP); Hideki Hokazono, Oita (JP); Mihoko Furutera, Oita (JP); Yasufumi Umemoto, Oita (JP); Kei Hayashi, Oita (JP); Naoki Takeshima, Oita (JP); Hideki Ohba, Saga (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignees: SANWA SHURUI CO., LTD., Oita (JP); BARLEY FERMENTATION TECHNOLOGIES, INC., Oita (JP)

(21) Appl. No.: 12/088,186

(22) PCT Filed: Sep. 26, 2006
(86) PCT No.: PCT/JP2006/319016

§ 371 Date: Jun. 26, 2009

(30) Foreign Application Priority Data
Sep. 26, 2005 (JP) 2005/277652
Sep. 26, 2005 (JP) 2005/277653

(51) Int. Cl. A61K 35/74; A61K 36/8998; A61P 9/00

(52) U.S. Cl. 424/93.4; 424/750

(57) ABSTRACT

It is an object of the invention to provide a composition with an excellent action of inhibiting vascularization, as prepared by a simple method suitable for actual production from an inexpensive, relatively readily available material without any problematic safety profile at an industrial scale without any complicated purification step. The composition is a composition for inhibiting vascularization, which contains a barley-derived ingredient selected from the group consisting of an unpolished barley ethyl alcohol extract fraction, an unpolished barley alkali extract fraction and fermented barley (preferably, a residual solution from the distillation of barley distilled spirits) as the active component with an action of inhibiting vascularization. The composition for inhibiting vascularization is a composition for therapeutically treating or preventing diseases which vascularization should be inhibited, specifically diseases with an etiology of abnormal vascularization in tumor or cancer, chronic inflammation or retinopathy. The composition is in a form selected from the group consisting of food additives, food materials, foods and drinks, pharmaceutical products under regulations by the Ministry of Health and Labor in Japan and quasi-pharmaceutical products under regulations by the Ministry of Health and Labor in Japan, and feeds.

### TECHNICAL FIELD

The present invention relates to a composition containing an ingredient derived from cereals and having an action of inhibiting vascularization. More specifically, the invention relates to a composition for therapeutically treating or preventing diseases that vascularization should be inhibited, the composition containing a substance contained in barley, preferably an unpolished barley fraction extracted in ethyl alcohol, an unpolished barley fraction extracted in alkalis, or an ingredient derived from fermented barley [preferably, a substance contained in a residual solution from the distillation of barley distilled spirits (a Japanese clear liquor), more preferably a composition (fermented barley extract) obtained by fractionating a residual solution from the distillation of barley distilled spirits] as the active component. Still more preferably, the invention relates to such composition in a form of, for example, food materials, foods and drinks, and feeds.

### BACKGROUND OF THE INVENTION

Cancer has been the disease at the highest rank of mortality causes in Japan since 1981. Only patients with cancer among the three major death causes increase consistently, involving the increase of the number of the dead patients therefor. According to the demographic statistics by the Ministry of Health and Labor, 300,658 individuals died due to the cause of cancer, among the 970,331 dead individuals in total number in 2001, which indicates actually that one of 3 died of cancer. For such diseases, principally, individual therapies such as pharmaceutical, chemical and physical treatments are currently practiced as symptomatic treatments in hospitals to directly attack cancer cells. These methods damage cancer cells themselves but also disadvantageously damage normal cells, leading to the deterioration of immunopotencies and spontaneous cure potencies, so that the patients consequently die very frequently. In such circumstances, a demand toward a safe therapeutic method with less adverse actions has been increasingly elevated. Attention is significantly focused on the prevention of diseases rather than the therapeutic treatments of diseases.

In recent years, the application of the inhibition of vascularization as a safe therapeutic method of cancer has been drawing attention. Cancer cells characteristically generate vascularization-promoting substances to spread vascular pathways for distributing nutrients and oxygen to their cells so as to continuously supply blood sufficiently and repeat their proliferation in an explosive manner. By the cancer therapy to which the action of inhibiting vascularization is applied, the blood pathway to cancer cells is blocked to prevent the proliferation of cancer cells. In other words, the cancer therapy can be defined simply as cutting off the supply of nutrients and oxygen to cancer cells.

Reports have been issued recently that the action of suppressing vascularization is effective in preventing and therapeutically treating safely not only cancer but also various diseases such as rheumatic arthritis, diabetic mellitus and heart diseases (Non-patent references 1 to 3). It is strongly desired to provide a substance with an action of suppressing vascularization in very near future.

As active ingredients with an action of inhibiting vascularization from natural origins, Patent reference 1 describes vascularization inhibitors, cell proliferation inhibitors, inhibitors of luminal formation and FGF inhibitors containing tocotrienol obtained from fruit skins and seeds of plants of the family palm as the active ingredients, as well as foods or food additives containing the same. Patent reference 2 describes food compositions containing shiitake mushroom mycelia extract for inhibiting vascularization. Patent references 3 and 4 describe that a novel compound from a liquid culture prepared by culturing a fungal microorganism has an action of inhibiting vascularization.

Meanwhile, barley as one plant species of the family Gramineae has been a cereal inevitable for humans since prehistoric times, and has been familiar as a highly healthy food as described in, for example, traditional medical textbooks in Japan. Actually, various research works have been done about the physiological functions of barley and barley fermented with a microorganism.

Patent reference 5 describes that via extraction of barley species crushed explosively in an aqueous solvent, the resulting extract mainly from a cereal skin fraction has useful physiological actions, namely immunoenhancement action, hypotensive action, blood circulation-ameliorating action, angiotension I transferase-inhibiting action, and antibacterial action, and also describes functional food materials utilizing the extract. However, the invention relates to the extract from the cereal skin fraction of barley, whose the active ingredient is a substance readily soluble in water, of a molecular weight of 500,000 or less as the whole and a molecular weight of 100,000 or less as the main ingredient, at a protein content of 3 to 30%, and with abundance in water-soluble ferulic acid and p-coumaric acid.

Patent reference 6 describes as an invention utilizing a residual solution from the distillation of barley distilled spirits produced as a by-product in producing distilled spirits from a raw material barley that an ethanol-insoluble fraction containing organic acids, protein and hemicellulose is prepared by obtaining a liquid fraction from a residual solution from the distillation of barley distilled spirits by solid-liquid separation, adding an alkali to the liquid fraction to fractionate an alkali-soluble fraction, neutralizing the alkali-soluble fraction with an acid to obtain a neutral soluble fraction and adding ethanol to the neutral soluble fraction and that the ethanol-insoluble fraction has an action of suppressing fat liver. Parent reference 7 describes that an organic solvent-insoluble fraction obtained by first preparing a liquid fraction from a residual solution from the distillation of barley distilled spirits via solid-liquid separation and then adding an organic solvent to the liquid fraction has an action of inhibiting leukemia cell proliferation. Patent reference 8 describes that the organic solvent-insoluble fraction obtained from a residual solution from the distillation of barley distilled spirits in the same manner as in Patent reference 7 activates natural killer cell.

Patent reference 9 describes a food composition comprising a non-adsorbed fraction prepared by obtaining a liquid fraction from a residual solution from the distillation of barley distilled spirits via solid-liquid separation, and then subjecting the liquid fraction to a synthetic adsorbent to separate the non-adsorbed fraction, which contains plural types of peptides of a mean chain length of 3.0 to 5.0 and with an amino acid composition of glutamic acid at 24 to 38%, glycine at 4 to 20%, aspartic acid at 5 to 10%, proline at 4 to 9% and serine at 4 to 8% in the total content of amino acids derived from the peptides as defined as 100%, where the food composition has an action of suppressing the onset of alcoholic liver damages and an action of curing alcoholic liver damages and has excellent taste, as well as a method for producing the food composition. Patent reference 10 describes that a composition obtained in the same manner as in Patent reference 9 is prepared as a pharmaceutical composition with an action of suppressing the onset of alcoholic liver damages and an action of curing the liver damages, as well as a method for producing the pharmaceutical composition.

Patent reference 11 describes that an organic solvent-insoluble fraction obtained by preparing a liquid fraction from a residual solution from the distillation of barley distilled spirits via solid-liquid separation, subjecting then the liquid fraction to an ion exchange treatment to obtain a fraction never adsorbed onto the ion exchange resin, subjecting the fraction never adsorbed onto the ion exchange resin to an ultrafiltration step to obtain a concentrated solution and then adding an organic solvent to the concentrated solution to separate an organic solvent-insoluble fraction has an antioxidative action.

Non-patent reference 1: Eur. J. Cancer, 32A, 2534-2539 (1996)
Non-patent reference 2: Nature Med., 1, 27-33 (1995)
Non-patent reference 3: Immunity, 12, 121 (2000)
Patent reference 1: JP-A-2002-308768
Patent reference 2: JP-A-2004-196791
Patent reference 3: JP-A-2003-183249
Patent reference 4: JP-A-2004-262881
Patent reference 5: JP-A-2002-371002
Patent reference 6: JP-A-2001-145472
Patent reference 7: JP-A-2003-73294
Patent reference 8: JP-A-2003-73295
Patent reference 9: JP-A-2004-112
Patent reference 10: JP-A-2004-2266
Patent reference 11: JP-A-2004-238452

### DISCLOSURE OF THE INVENTION

### Problems that the Invention is to Solve

As described above, it has been revealed that barley and fermented barley have various physiological activities. However, the action of inhibiting vascularization is not included in the physiological activities. It has never been known that barley has an action of inhibiting vascularization and that barley can exert the action more strongly when barley is fermented.

It is an object of the invention to provide a composition with an excellent action of inhibiting vascularization, as prepared by a simple method suitable for actual production from an inexpensive, relatively readily available material without any problematic safety profile (a plant of the family Gramineae), preferably from barley, more preferably from fermented barley, at an industrial scale without any complicated purification step. Preferably, the composition is a composition for therapeutically treating or preventing diseases that vascularization should be inhibited. More specifically, it is an object of the invention to provide such composition in a form selected from the group consisting of food additives, food materials, foods and drinks, pharmaceutical products under regulations by the Ministry of Health and Labor in Japan and quasi-pharmaceutical products under regulations by the Ministry of Health and Labor in Japan, and feeds.

### Means for Solving the Problems

So as to overcome the problems, the inventors made investigations. Consequently, the inventors found that a cereals-derived ingredient, particularly a barley-derived ingredient has an excellent action of inhibiting vascularization and that the action is more effective when barley is fermented. In accordance with the invention, an unpolished barley fraction extracted in ethyl alcohol (an unpolished barley ethyl alcohol extract fraction) and an unpolished barley fraction extracted in alkalis (an unpolished barley alkali extract fraction) have significant actions of inhibiting vascularization, so a new composition with an activity of inhibiting vascularization and with less adverse actions can be provided. In accordance with the invention, further, a composition obtained by fractionating a residual solution from the distillation of barley distilled spirits has a significant action of inhibiting vascularization, so a new composition with an activity of inhibiting vascularization and with less adverse actions can be provided.

The invention relates to the following compositions for inhibiting vascularization as described below in (1) through (13).

(1) A composition for inhibiting vascularization characterized by containing an ingredient derived from a plant of the family Gramineae as the active component.
(2) A composition for inhibiting vascularization as described above in (1), where the ingredient derived from a plant of the family Gramineae is an ingredient derived from barley.
(3) A composition for inhibiting vascularization as described above in (2), where the ingredient derived from barley is selected from unpolished barley ethyl alcohol extract fractions and unpolished barley alkali extract fractions.
(4) A composition for inhibiting vascularization as described above in (3), where the ingredient derived from barley is an ingredient derived from fermented barley.
(5) A composition for inhibiting vascularization as described above in (4), where the ingredient derived from fermented barley is a residual solution from the distillation of barley distilled spirits.
(6) A composition for inhibiting vascularization as described above in (4) or (5), where the ingredient derived from fermented barley is selected from the group consisting of a fermented barley extract and fermented barley fiber.
(7) A composition for inhibiting vascularization as described above in (6), where the fermented barley extract is selected from a composition obtained by fractionating a residual solution from the distillation of barley distilled spirits, a lactic acid bacterium liquid culture in a culture medium of the residual solution from the distillation of barley distilled spirits and a Natto-kin (a Bacillus subtilis species working for preparing natto (fermented soybean product) liquid culture in a culture medium of the residual solution from the distillation of barley distilled spirits and where the fermented barley fiber is a fermented barley fiber extracted in alkalis.
(8) A composition for inhibiting vascularization as described above in (7), where the composition obtained by fractionating a residual solution from the distillation of barley distilled spirits is one or more compositions selected from the group consisting of a non-adsorbed fraction of the residual solution from the distillation of barley distilled spirits, which is never adsorbed on a synthetic adsorbent, an adsorbed fraction of the residual solution from the distillation of barley distilled spirits, which is adsorbed on a synthetic adsorbent, and a fraction of the residual solution from the distillation of barley distilled spirits, which is preliminarily precipitated with ethanol.
(9) A composition for inhibiting vascularization as described above in any one of (1) through (8), where the composition for inhibiting vascularization is a composition for therapeutically treating or preventing a disease that vascularization should be inhibited.
(10) A composition for inhibiting vascularization as described above in (9), where the disease that vascularization should be inhibited is a disease with an etiology of abnormal vascularization in tumor or cancer, chronic inflammation or retinopathy.
(11) A composition for inhibiting vascularization as described above in any one of (1) through (10), where the composition is in a form selected from food additives, food materials, foods and drinks, pharmaceutical products defined under the regulations by the Ministry of Health and Labor in Japan and quasi-pharmaceutical products defined under the regulations by the Ministry of Health and Labor in Japan, and feeds for inhibiting vascularization.
(12) A composition for inhibiting vascularization as described above in (11), where the foods and drinks are functional foods, nutritional supplement foods or health foods and drinks for inhibiting vascularization.
(13) A composition for inhibiting vascularization as described above in (11), where the feeds are feeds for cattle, poultry, and pets for inhibiting vascularization.

### ADVANTAGES OF THE INVENTION

In accordance with the invention, there can be provided a composition with an excellent action of inhibiting vascularization with less adverse actions, as prepared by a simple method suitable for actual production from an inexpensive, relatively readily available material without any problematic safety profile (unpolished barley), preferably from fermented barley, more preferably a residual solution from the distillation of barley distilled spirits, at an industrial scale without any complicated purification step. Preferably, the composition is a composition for therapeutically treating or preventing diseases that vascularization should be inhibited. In accordance with the invention, more preferably, there can be provided such composition in a form selected from the group consisting of food additives, food materials, foods and drinks, pharmaceutical products defined under the regulations by the Ministry of Health and Labor in Japan and quasi-pharmaceutical products defined under the regulations by the Ministry of Health and Labor in Japan, and feeds.

### BRIEF DESCRIPTIONS OF THE DRAWINGS

FIG. 1 shows microscopic photographs depicting the appearance of luminal formation with VEGF-A and suramin at 50 μM in Example 1 in place of drawings, where A1 and A2 are photographs with a negative control VEGF-A and A3 and A4 are photographs with a positive control suramin.

FIG. 2 shows microscopic photographs depicting the appearance of luminal formation with the unpolished barley ethanol extract in Example 1 and with a control with no additives in place of drawings, where B1, B2 and B3 are photographs with the unpolished barley ethanol extract added at 10 μg, 100 μg and 1,000 μg, respectively while B4 is a photograph with a control with the culture medium alone without any such ethanol extract added.

FIG. 3 shows microscopic photographs depicting the appearance of luminal formation with the unpolished barley alkali extract in Example 1 and with a control with no additives in place of drawings, where C1 is a photograph with the unpolished barley alkali extract added at 10 μg, while C4 is a photograph with a control with the culture medium alone without any such alkali extract added.

FIG. 4 shows bar graphs describing the results of vascularization inhibition tests in Example 2.

FIG. 5 shows photographs depicting the influence of fermented barley extract in Example 3 on vascularization in place of drawings.

FIG. 6 shows microscopic photographs depicting the appearance of luminal formation with the fermented barley extract powder P in Example 4 and with a control with no such powder added in place of drawings, where the appearance of luminal formation with VEGF-A or suramin at 50 μM or with the fermented barley extract powder P is shown. Appearance of luminal formation using VEGF-A and suramin at 50 μM (from FIG. 1).

Negative controls A1 and A2: VEGF-A
Positive controls A3 and A4: suramin
Fermented barley extract powder P (at an amount added): D1 at 10 μM and D4 with only the culture medium added.

FIG. 7 shows bar graphs describing the results of vascularization inhibition tests in Example 5.

FIG. 8 shows bar graphs depicting the influence on the weight of granulated tissues.

FIG. 9 shows bar graphs depicting the influence on the hemoglobin concentration in granulated tissues.

FIG. 10 shows bar graphs depicting the influence on the ratio of the vascular tube area in granulated tissues.

FIG. 11 shows microscopic photographs depicting the histopathological images of granulated tissues in individual sample-dosed groups 2 weeks after sponge planting, where (A) shows a control group; (B) shows a group dosed with 1% fermented barley extract powder; (C) shows a group dosed with 5% fermented barley extract powder; (D) shows a group dosed with 1% fermented barley extract powder P; and (E) shows a group dosed with 1% unpolished barley ethanol extract fraction powder, where the symbol ▴ represents vascularization.

### BEST MODE FOR CARRYING OUT THE INVENTION

The composition with an activity of inhibiting vascularization in accordance with the invention may be prepared from plants of the family Gramineae, for example, barley, wheat, rye, oat, adlay and rice. Preferably, barley is used. Barley may be any of hulled barley and hulless barley or may be any of two-raw barley or six-raw barley. Additionally, barley may be colored barley that dyes are deposited on the cereal surface. As the cereal form, any of the whole cereal grain, the surface skin part thereof, and the endosperm thereof may be used with no specific limitation. Preferably, the whole cereal grain such as unpolished barley may be used. Additionally, the cereal may be treated with processes such as roasting, pulverizing, and pressuring processes. In case of the fermentation of barley, the barley as the fermentation subject is as described above.

Any microorganism may be used with no limitation for barley fermentation. Yeast, lactic acid bacterium, Natto-kin or koji may be used. As the yeast for use in the fermentation process in preparing fermented barley extracts, in particular, beer yeast, Japanese sake yeast, distilled spirits yeast, wine yeast and bakery's yeast may be included. Preferably, distilled spirits yeast is used.

The composition of the invention can be prepared by a simple method suitable for actual production from an inexpensive, relatively readily available material without any problematic safety profile (a plant of the family Gramineae), preferably from barley, more preferably fermented barley, at an industrial scale without any complicated purification step. The application thereof to biological organisms may be done in forms of foods and drinks, pharmaceutical products, fertilizers, feeds and dermal external agents, so that an excellent effect on inhibiting vascularization can be expected.

The composition of the invention contains an active ingredient derived from naturally occurring materials with an action of inhibiting vascularization. Via the application of the vascularization inhibition based on the active ingredient, for example, a safe therapeutic method of cancer can be done. The action of suppressing vascularization is effective in preventing and therapeutically treating safely not only cancer but also various diseases such as rheumatic arthritis, diabetes mellitus, and heart diseases. Thus, the composition of the invention is a functional composition.

In other words, the composition of the invention is a composition for therapeutically treating or preventing a disease that vascularization should be inhibited. The disease that vascularization should be inhibited includes any diseases that vascularization works significantly for the occurrence of pathological conditions. Therefore, the disease which vascularization should be inhibited includes diseases with an etiology of abnormal vascularization in tumor or cancer, or chronic inflammation or retinopathy. More specifically, the disease that vascularization should be inhibited includes, for example, but is not limited to tumor or cancer such as solid tumor emerging in various tissues, myeloma and angioma; chronic inflammation such as chronic rheumatoid arthritis, psoriasis, and osteoarthritis; or age-related macular degeneration, diabetic retinopathy and neovascular glaucoma. In accordance with the invention, target diseases for which vascularization should be inhibited are preferably various tumor types and cancer types.

So as to examine in vitro the activity of the composition of the invention for inhibiting vascularization, vascular endothelial cells and fibloblast cells are co-cultured in the presence of the vascular endothelial growth factor VEGF if necessary, with the addition of the composition of the invention when the culture is promoted to a growth stage in an early stage of luminal formation, so as to verify whether or not vascularization is suppressed. The suppression of vascularization can be examined by staining the lumen to examine the suppression of luminal formation. As a kit for examining in vitro the effect on the inhibition of vascularization, a vascularization kit (manufactured by KURABO) can be used; for staining lumen, a lumen staining kit (manufactured by KURABO) with antibodies such as anti-CD31 antibody and an anti-von Willebrand factor antibody can be used.

The composition of the invention characteristically contains a barley-derived ingredient as the active component for the action of inhibiting vascularization. The substance exerting the action of inhibiting vascularization as contained in the barley-derived ingredient is a substance contained in preferably unpolished barley ethanol extract fractions and unpolished barley alkali extract fractions.

Furthermore, the composition of the invention characteristically contains a fermented barley-derived ingredient as the active component for the action of inhibiting vascularization. The substance exerting the action of inhibiting vascularization as contained in the fermented barley-derived ingredient is a substance contained in preferably residual solution from the distillation of barley distilled spirits. The substance contained in the residual solution from the distillation of barley distilled spirits is a substance contained in a composition selected from the group consisting of a composition obtained by fractionating the residual solution from the distillation of barley distilled spirits (fermented barley extract), a lactic acid bacterium liquid culture in a culture medium of the residual solution from the distillation of barley distilled spirits, a Natto-kin liquid culture in a culture medium of the residual solution from the distillation of barley distilled spirits, and a fermented barley fiber alkali extract.

The method for obtaining the unpolished barley ethanol extract fraction includes, for example, a method comprising pulverizing unpolished barley with a mill, adding ethanol to the resulting powder for extraction, filtering the extract solution with a filter paper, concentrating the resulting filtrate under pressure while removing ethanol, and centrifuging the concentrate to recover a liquid fraction from the unpolished barley ethanol extract solution. The method for obtaining the unpolished barley alkali extract fraction includes, for example, a method comprising pulverizing unpolished barley with a mill, adding an aqueous alkali solution to the resulting powder to obtain an extract solution, neutralizing the extract solution with an acid, and continuously filtering the resulting solution with a filter paper, to obtain the liquid fraction of the unpolished barley alkali extract solution.

The method for obtaining the composition from the fractionation of the residual solution from the distillation of barley distilled spirits includes a method comprising solid-liquid separation with a screw press to obtain the solid, and a method comprising purifying the liquid fraction obtained via the solid-liquid separation on a column packed with a synthetic adsorbent or an ion exchange resin, subsequently freeze-drying the resulting purified product or preparing the purified product as an insoluble matter with an organic solvent. Additionally, a method is listed, comprising using the liquid fraction obtained via solid-liquid separation in a culture medium for culturing a lactic acid bacterium or Natto-kin, and then freeze-drying the resulting liquid culture. Therefore, the composition obtained via the fractionation of the residual solution from the distillation of barley distilled spirits is, for example, one or more compositions selected from the group consisting of a non-adsorbed fraction of the residual solution from the distillation of barley distilled spirits, which is never adsorbed on a synthetic adsorbent, an adsorbed fraction of the residual solution from the distillation of barley distilled spirits, which is adsorbed on a synthetic adsorbent, a non-adsorbed fraction of the residual solution from the distillation of barley distilled spirits, which is treated with the ion exchange resin and a fraction of the residual solution from the distillation of barley distilled spirits, which is preliminarily precipitated with ethanol.

The composition is obtained by the fractionation of the residual solution from the distillation of barley distilled spirits through the following steps. First, barley koji is produced. After water is absorbed in barley to 40 w/w %, which is then steamed for 40 minutes, the resulting barley is left to cool to 40° C., to which seed koji (white koji species) is inoculated at a ratio of 1 kg to 1 ton of barley for allowing the barley to be left at 38° C. and 95% RH for 24 hours and then at 32° C. and 92% RH for 20 hours, so that barley koji can be produced. To the barley koji produced by the method, water and cultured bacterial cells of yeast for distilled spirits are added to prepare a primary moromi, which is then subjected to fermentation for 5 days (fermentation at first stage). At the second charging, then, water and the barley koji produced by the method are added to the primary moromi on completion of the fermentation at the first stage, for 11-day fermentation (fermentation at the second stage). The fermentation temperature is 25° C. for both the first charging and the second charging. The secondary moromi on completion of the fermentation at the second stage is subjected to single distillation, to obtain barley distilled spirits and a residual solution from the distillation of barley distilled spirits at a koji yield of 100%.

A solid fraction obtained by the solid-liquid separation of the residual solution from the distillation of barley distilled spirits may be used as it is or a liquid fraction obtained in the same manner is mixed with dextrin and then freeze-dried (fermented barley extract powder) for use. Otherwise, the liquid fraction is allowed to pass through a column packed with a synthetic adsorbent, so that the resulting non-adsorbed fraction (fermented barley extract powder S) may be used while a fraction obtained by eluting the adsorbed fraction may also be used (fermented barley extract powder P) Additionally, the fraction may be treated with an organic solvent to prepare the fraction in an insoluble matter, so that the resulting insoluble fraction (ethanol-precipitated fraction powder) may also be used. Before the treatment with an organic solvent, furthermore, the fraction may be passed through a column packed with an ion exchange resin; otherwise, the fraction may be passed through a column with an ion exchange resin and may then be passed through an ultrafiltration filter membrane, for use.

As the synthetic adsorbent for the aforementioned use, there may be used synthetic adsorbents of aromatic series, modified aromatic series or methacrylic series. Preferably, specific examples of such synthetic adsorbent include Amberlite XAD-4, Amberlite XAD-16, Amberlite XAD-1180 and Amberlite XAD-2000 manufactured by Organo; aromatic (or styrenic) synthetic adsorbents such as Sepabeads SP850 and Dia-ion HP20 manufactured by Mitsubishi Chemical Co., Ltd.; methacrylic-series (or acrylic-series) synthetic adsorbents such as Amberlite XAD-7 manufactured by Organo and Dia-ion HP2MG manufactured by Mitsubishi Chemical Co., Ltd.; and synthetic adsorbents of modified aromatic series, such as Sepabeads SP207 manufactured by Mitsubishi Chemical Co., Ltd. Preferable specific examples of the ion exchange resin for the aforementioned use include strongly acidic cation exchange resins manufactured by Organo, such as IR-120, IR-120B and Amberlite 200CT and poorly acidic cation exchange resins manufactured by Organo, such as IRC50 and IRC76; strongly acidic cation exchange resins Dia-ion manufactured by Mitsubishi Chemical Co., Ltd. such as SK1B, SK104 and PK208 and poorly acidic cation exchange resins manufactured by Mitsubishi Chemical Co., Ltd. such as WK10 and WK40.

The organic solvent for the aforementioned use is preferably ethanol, which is added most preferably to 75% by volume as the final concentration.

As an additional mode, a residual solution from the distillation of barley distilled spirits is used in a culture medium for a lactic acid bacterium and Natto-kin, to obtain a liquid culture, which is then freeze-dried (the powder of the lactic acid bacterium liquid culture, the powder of the Natto-kin liquid culture). In other words, the fermented barley extract is a composition obtained by fractionating the residual solution from the distillation of barley distilled spirits and the residual solution from distillation of barley distilled spirits and is additionally selected from the group consisting of a lactic acid bacterium liquid culture in a culture medium of the residual solution from the distillation of barley distilled spirits, a Natto-kin liquid culture in a culture medium of the residual solution from the distillation of barley distilled spirits, and a fermented barley fiber alkali extract.

The lactic acid bacterium for the aforementioned use preferably includes lactic acid bacteria of Lactococcus lactis subsp. Lactis but is not limited. Specifically, the lactic acid bacterium includes Lactococcus lactis NCDO 497, Lactococcus lactis NIZO R5, Lactococcus lactis ATCC 7962 and Lactococcus lactis ATCC 11454, Lactococcus lactis NIZO 22186, Lactococcus lactis NRRL-B-18583, Lactococcus lactis NCFB 2118, Lactococcus lactis NCFB 2054, Lactococcus lactis NIZO N9, Lactococcus lactis NIZO 221186, Lactococcus lactis IO-1 (JCM 7638), Lactococcus lactis subsp. Lactis A. Ishizaki Chizuka (JCM 11180), Lactococcus lactis subsp. Lactis A. Ishizaki Yasaka 5B (JCM 11181), Lactococcus lactis subsp. Lactis A. Ishizaki Yasaka 7B (JCM 11182), Lactococcus lactis subsp. Lactis A. Ishizaki Yasaka 8B (JCM 11183), and Lactococcus lactis subsp. Lactis A. I.

The Natto-kin includes the Miyagino strain as one commercially available Natto-kin species belonging to Bacillus subtilis but is not limited.

The composition containing the unpolished barley ethanol extract, the unpolished barley alkali extract fraction, fermented barley and/or a residual solution from the distillation of barley distilled spirits is in a form selected from the group consisting of food additives, food materials, foods and drinks, pharmaceutical products defined under the regulations by the Ministry of Health and Labor in Japan and quasi-pharmaceutical products defined under the regulations by the Ministry of Health and Labor in Japan and feeds for inhibiting vascularization. Active utilization of the functionality of the composition enabled the development of healthy foods and drinks, foods as nutritional foods and drinks for patients, and feeds for feeding animals such as cattle, poultry and fish.

Specifically, the foods and drinks are functional foods and drinks, nutritional supplement foods or healthy foods and drinks for inhibiting vascularization. The feeds are feeds for cattle, poultry and pets for inhibiting vascularization. Specifically, food materials containing the unpolished barley ethanol extract, the unpolished barley alkali extract fraction, the fermented barley and/or the residual solution from the distillation of barley distilled spirits may be used in any form of food forms, drink forms or feed forms.

When the composition for inhibiting vascularization in accordance with the invention is used for actively utilizing the functionality thereof, the content thereof is not specifically limited but may appropriately be adjusted, depending on the level of an intended function, the mode for use, and the amount thereof used. For example, the content is 0.001 to 100% by mass. The composition for inhibiting vascularization may be used for humans and additionally as foods and drinks, pharmaceutical products under the regulations by the Ministry of Health and Labor in Japan, feeds and dermal external agents. The composition may be dosed orally or may be coated on skin and the like. According to general methods, the composition is blended in oral products or parenteral products and may be used in various fields of seasonings, food additives, food materials, foods and drinks, healthy foods and drinks, dermal external agents, pharmaceutical agents under regulations by the Ministry of Health and Labor in Japan, and feeds. When the composition is blended for example in a food or a drink, the resulting food or the resulting drink may provide a food or a drink for therapeutically treating or preventing a disease that vascularization should be inhibited. From the standpoint of prophylactic effects the composition may be promising for use for example as a healthy food or a nutritional food. Additionally, the composition may be used as feeds or foods for cattle and/or fishes. By using the composition for humans or as foods and drinks, pharmaceutical products under the regulations by the Ministry of Health and Labor in Japan, fertilizers, feeds and dermal external agents, diseases that vascularization should be inhibited can be effectively treated therapeutically or prevented.

The composition may readily be prepared from unpolished barley, preferably from the cost standpoint and from the aspect of efficacious resource application.

The composition of the invention for use in foods may be used as it is, or may be prepared in a form diluted with oil or the like, or in an emulsion form of meal or in forms with added carriers for general use in food industries.

Those in an emulsion form can be prepared for example by adding the composition to an oil phase part, further adding liquid fats such as glycerin fatty acid esters, sorbitan fatty acid esters, sucrose fatty acid esters, glycerol, dextrin, rapeseed oil, soybean oil, and corn oil, adding L-ascorbic acid or an ester or a salt thereof, adding for example rubber materials such as locust bean gum, gum Arabic or gelatin, flavonoids or polyphenols such as hesperidin, rutin, quercetin, catechin, and thianidine or mixtures with the flavonoids or polyphenols, and then emulsifying the resulting mixtures.

The drink form is a non-alcohol drink or an alcohol drink. The non-alcohol drink includes for example carbonate drinks, non-carbonate drinks such as fruit juice drinks and nectar drinks, refreshing drinks, sports drinks, tea, coffee and cocoa; and the alcohol drink forms includes for example forms of general food products, such as spirits, liquors, distilled spirits drinks, wines, beer, low-malt beer and pharmaceutical liquors.

The drinks and foods specifically include for example those described below: cakes and pastries (pudding, jelly, goumi candy, candy, drops, caramel, chewing gum, chocolate, pastry, butter cream, custard cream, cream puff, hot cake, bread, potato chip, fried potato, pop corn, biscuit, cracker, pie, sponge cake, pao de Castella, waffle, cake, doughnut, cookies, rice cracker, small rice cracker, sweat rice cracker, bun with bean-jam filling, and candies); dried noodle products (macaroni, pasta), egg products (mayonnaise, fresh cream); drinks (functional drinks, lactic acid drinks, yoghurt drinks, concentrated milk-derived drinks, fruit juice drinks, non-fruit juice drinks, fruit flesh drinks, clear carbonate drinks, fruit juice-added carbonate drinks, fruit flesh-colored carbonate drinks); tasting products (green tea, black tea, instant coffee, cocoa, canned coffee drinks); milk products (ice cream, yoghurt, coffee milk, butter, butter sauce, cheese, fermented milk, processed milk); pastes (marmalade, jam, flower paste, peanut paste, fruit paste, fruit flesh dipped in syrup); cattle products (ham, sausage, bacon, dry sausage, beef jerky, lard); fishes and shell fishes (fish ham, fish sausage, fish cake, hollowed fish cakes in tubular shape, cake of pounded fish, dried and salted fish, dried bonito, dried mackerel, small dried sardines, sea urchin, salted squid organ, dried squid, dried fish after dipping in sweet sake, dried shell, smoked products of fishes such as salmon); fishes, shell fishes and seaweeds boiled with seasonings such as soy sauce and sugar (small fishes, shell fishes, edible wild plants, mushroom, kelp); curries (instant curries, retorte curries, canned curries); seasonings (miso paste, powdery miso, soy sauce, powdery soy sauce, moromi (unrefined sake or soy sauce), fish-derived sauce tasting like soy sauce, Worcestershire sauce, ketchup, oyster sauce, solid bouillon, groovy for broiled meat, curry roux, stew mix, soup mix, seasoning mix for Japanese soup, paste, instant soup, toppings, dressing, salad oil); fried products (oil fries, oil-fried confectioneries, instant Chinese noodle); soybean milk, margarine, and shortening.

The foods and drinks described above can be produced by blending the composition with raw materials for general foods and then processing the resulting blend.

The amount of the composition to be blended in the foods and drinks may vary, depending on the form of a food product, with no specific limitation. Generally, the amount thereof is preferably 0.001 to 20%.

The foods and drinks may also be used as functional foods, nutritional supplement foods or healthy foods. The form thereof is not specifically limited. In a production example of a food, proteins such as milk protein, soybean protein and egg albumin with high nutritional values and in good balance of amino acids, decomposed products thereof, egg white oligopeptides, processed soybean products and mixtures of single amino acids may be used according to general methods. Additionally, the foods and drinks may be utilized in forms of for example soft capsules and tablets.

Examples of the nutritional supplement foods or functional foods include processed forms such as fluid meals, semi-digested nutrient meals, component nutrient meals, drinks, capsules, and enteral nutrients, in blend with sugars, fats, trace elements, vitamins, emulsifying agents and flavor. The various foods described above, for example the drinks and foods such as sports drink and nutritious drink may be blended additionally with nutritious additives such as amino acids, vitamins and minerals, sweeteners, spices, flavor and dyes so as to improve the nutritious balance and the flavor.

So as to stabilize the composition of the invention, antioxidants for example tocopherol, L-ascorbic acid, BHA and rosemary extracts may be used concurrently with the composition according to general methods.

The composition of the invention may be applicable for feeds for cattle, poultry and pets. The composition of the invention may be blended into for example dry dog foods, dry cat foods, wet dog foods, wet cat foods, semi-moist dog foods, feeds for chicken feeding, and feeds for cattle including cow and pig. The feeds per se may be prepared according to general methods.

These therapeutic agents and preventive agents may also be used for animals other than humans, for example, cattle mammals such as cow, horse, pig and sheep, poultry such as chicken, quail, and ostrich, pets such as reptiles, birds or small mammals, and hatchery fishes.

The details of the invention are now described in Examples. The invention is never limited by these Examples. Specifically, there are now descriptions about the method for preparing each composition from unpolished barley as a raw material and the effect on inhibiting vascularization (Examples 1 and 2), the preparation of each composition from the liquid fraction in a residual solution from the distillation of barley distilled spirits as a material and the effect on inhibiting vascularization (Examples 3, 4 and 5), and the effect of the fermented barley extract, the fermented barley extract P, and the unpolished barley ethanol extract fraction on inhibiting vascularization (Example 6). However, the scope of the invention is never limited by these examples.

### Example 1

### Experiment 1

### Experiment on Vascularization Inhibition

### <Sample Names>

The names of the samples are shown in Table 1, where A1 and A2 express negative controls; A3 and A4 express positive controls; B1 to B3 express unpolished barley ethanol extract fraction powders; and C1 to C3 express unpolished barley alkali extract fraction powders.

### <Method for Preparing Samples for Use in the Experiment on Vascularization Inhibition>

From the respect of other experiments, the samples used in this experiment were originally powders but the powders were re-dissolved for use in the experiment. However, samples in liquid may satisfactorily be used in the experiment.

### [Unpolished Barley Ethanol Extract Fraction Powder]

One liter of 75 v/v % ethanol was added to 100 g of unpolished barley pulverized with a mill, for extraction under agitation at ordinary temperature for 6 hours. The resulting extract solution was filtered through a filter paper 4C manufactured by ADVANTEC. The resulting filtrate was concentrated under reduced pressure, to remove ethanol. Next, the resulting concentrate was centrifuged under a condition of 10,000 rpm for 10 minutes, to obtain the liquid fraction in the unpolished barley ethanol extract solution. The extract solution was freeze-dried.

### [Unpolished Barley Alkali Extract Fraction Powder]

One liter of an aqueous 2% Ca(OH)₂ solution was added to 100 g of unpolished barley pulverized with a mill, for extraction under agitation at ordinary temperature for 6 hours. Using HCL, then, the extract solution was adjusted to pH 7.0. Next, the extract solution was filtered through a filter paper 4C manufactured by ADVANTEC, to obtain the liquid fraction in the unpolished barley alkali extract solution. The extract solution was freeze-dried.

### <Preparation of Experimental Specimen, Experimental Method and Results>

### 1. Experimental Specimen

### 1-1. Sample Preparation

1-1-1. For experiment for inhibiting vascularization

1,000 μg of each specimen was weighed and dissolved in 1 ml of a culture medium. The resulting culture medium was sterilized by filtration (0.22 μm), diluted 10 fold (three times), to prepare samples at concentrations of 10 μg/ml to 1,000 μg/ml as shown in Table 1 (the contents of the test samples for use in assessing the effect on inhibiting vascularization). Similarly, the negative controls and positive controls as shown in Table 1 were prepared.

### 2. Experimental Method and Results

Human vascular endothelial cells and fibroblasts were co-cultured at their optimal concentrations. At the proliferation state at an early stage of luminal formation, the individual samples (1-1-1, the negative controls, the positive controls) were added for 11-day culturing (the culture media containing the samples were exchanged 4, 7 and 9 days later). Subsequently, luminal formation was observed with a microscope by staining the formed lumen-like network structure with a mouse anti-human CD31/goat anti-mouse IgG AlkP conjugate. So as to evaluate the effect on inhibiting vascularization, the formed lumen-like network structure was observed.

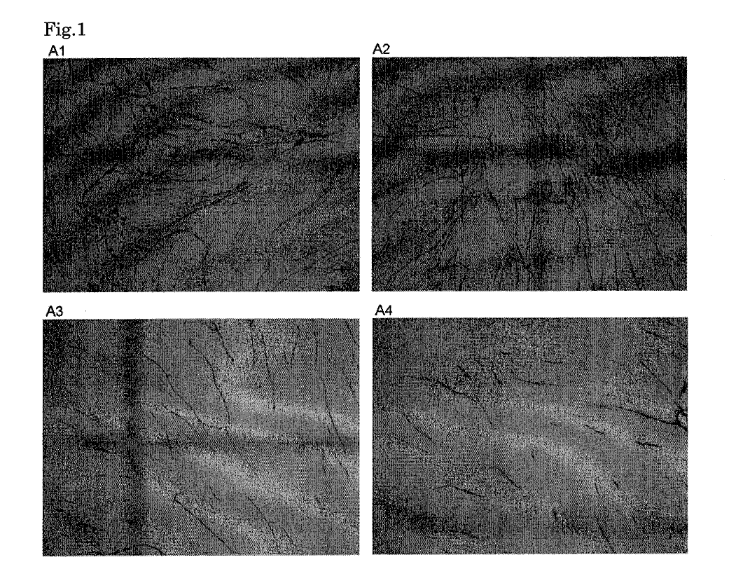

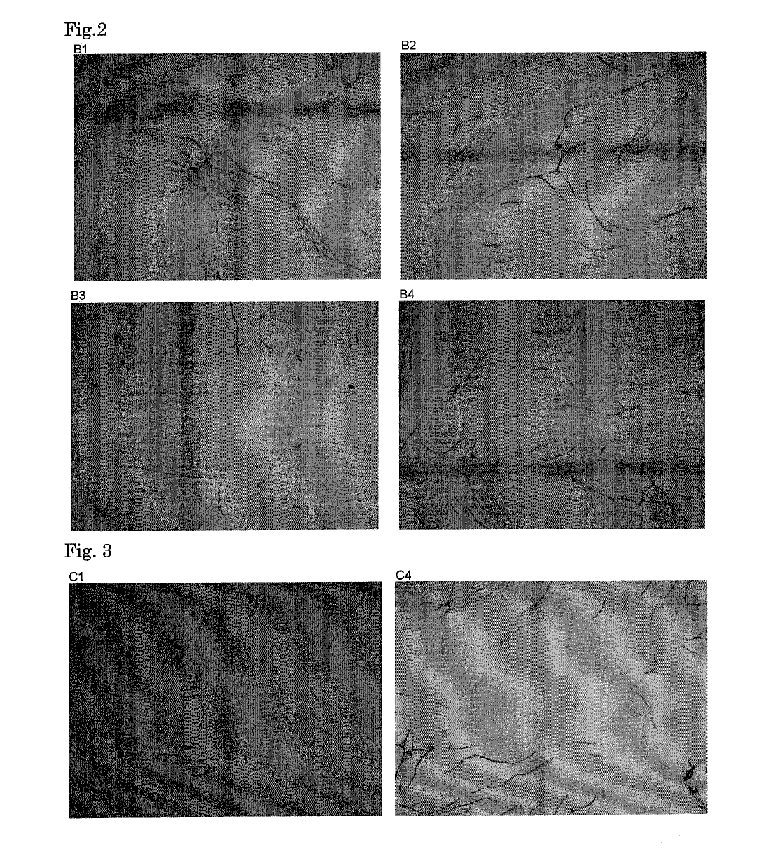

The results are shown in Table 2 and FIGS. 1 through 3. Photographs of the vascular tube tissues were incorporated as digital data, to measure the area of a vascular tube region (black region) at several points randomly selected to determine the effect on inhibiting vascularization. A smaller numerical figure in the column AREA in Table 2 shows a sample with a larger effect on inhibiting vascularization.

TABLE 1

| No. | Contents | Samples | |
|---|---|---|---|
| A1 | Negative | VEGF-A alone | |
| A2 | control | VEGF-A alone | |
| A3 | Positive | VEGF-A | suramin |
| A4 | control | VEGF-A | suramin |
| B1 | Test lot 1 | VEGF-A | Unpolished barley ethanol |
| | | | extract 10 μg |
| B2 | | VEGF-A | Unpolished barley ethanol |
| | | | extract 100 μg |
| B3 | | VEGF-A | Unpolished barley ethanol |
| | | | extract 1,000 μg |
| B4 | | Culture medium alone | |
| C1 | Test lot 2 | VEGF-A | Unpolished barley alkali extract |
| | | | 10 μg |
| C2 | | VEGF-A | Unpolished barley alkali extract |
| | | | 100 μg |
| C3 | | VEGF-A | Unpolished barley alkali extract |
| | | | 1,000 μg |

| | |
|---|---|
| C4 | Culture medium alone |

VEGF-A as a vascularization-promoting factor was added at an amount of 5 ng/ml (as the final concentration)
Suramin as a positive control with an observed effect on inhibiting vascularization was added at an amount of 25 μM (as the final concentration)

TABLE 2

| Sample | Microscopic | | | | |
|---|---|---|---|---|---|
| No. | photograph | AREA | LENGTH | JOINT | PATH |
| A1 | A1 in FIG. 1 | 76855 | 11872 | 69 | 267 |
| A2 | A2 in FIG. 1 | 92911 | 14727 | 96 | 351 |
| A3 | A3 in FIG. 1 | 27940 | 4583 | 11 | 107 |
| A4 | A4 in FIG. 1 | 33301 | 5311 | 16 | 128 |
| B1 | B1 in FIG. 2 | 52920 | 8533 | 40 | 194 |
| B2 | B2 in FIG. 2 | 32326 | 5644 | 18 | 120 |
| B3 | B3 in FIG. 2 | 17995 | 2433 | 6 | 82 |
| B4 | B4 in FIG. 2 | 39722 | 6937 | 18 | 129 |
| C1 | C1 in FIG. 2 | 34881 | 5827 | 32 | 160 |
| C4 | C4 in FIG. 2 | 45518 | 7640 | 42 | 173 |

An apparent effect on inhibiting vascularization was observed in the unpolished barley ethanol extract fraction.

### Example 2

### Experiment 2

### Experiment on Vascularization Inhibition

### <Sample Names>

The names of the samples are shown in Table 3.

### Method for Preparing Samples (1) Through (9) for Use in the Experiment on Vascularization Inhibition

From the respect of other experiments, the samples used in this experiment were originally powders but the powders were re-dissolved for use in the experiment. However, samples in liquid may satisfactorily be used in the experiment. As an excipient, dextrin at a volume equal to that of the extract was added to some of the samples during pulverization, as described below. Herein, the dextrin content is expressed in percentage by weight (w/w) concentration.

### (1) [Unpolished Barley Ethanol Extract Fraction Powder]

See the column 0044.

### (2) [Unpolished Barley Alkali Extract Fraction Powder]

See the column 0045.

### [Preparation of Experimental Specimen, Experimental Method and Results]

### 1. Experimental Specimen

### 1-1. Sample Preparation

### 1-1-1. For Experiment for Inhibiting Vascularization

1,000 μg of each specimen was weighed and dissolved in 1 ml of a culture medium. The resulting culture medium was sterilized by filtration (0.22 μm), diluted 10 fold (three times), to prepare samples at concentrations of 10 μg/ml to 1,000 μg/ml.

### 2. Experimental Method and Results

Human vascular endothelial cells and fibroblasts were co-cultured at their optimal concentrations. At the proliferation state at an early stage of luminal formation, the individual samples were added for 11-day culturing (the culture media containing the samples were exchanged 4, 7 and 9 days later). Subsequently, luminal formation was observed with a microscope by staining the formed lumen-like network structure with a mouse anti-human CD31/goat anti-mouse IgG AlkP conjugate. As standards for the evaluation, VEGF-A promoting luminal formation was added at 10 ng/ml, while suramin inhibiting luminal formation was added at 50 μM, in the same manner. A control was prepared without any addition. Regarding the effect on inhibiting vascularization, the formed lumen-like network structure was evaluated.

Method: A kit manufactured by KURABO was used for measurement. The lumen formation state (the area, the length and the number of branches) was observed at four points. VEGF as a vascularization promoting factor and suramin as an agent for suppressing vascularization were concurrently present in a control, so as to compare the lumen formation state in the co-existence of VEGF and each sample.

### (Comparison in Activity of Inhibiting Vascularization Between Suramin and Sample)

Concentration of a sample added: 10, 100 and 1,000 μg/ml (Suramin was added in all of the cases at the same concentration; 50 μM)

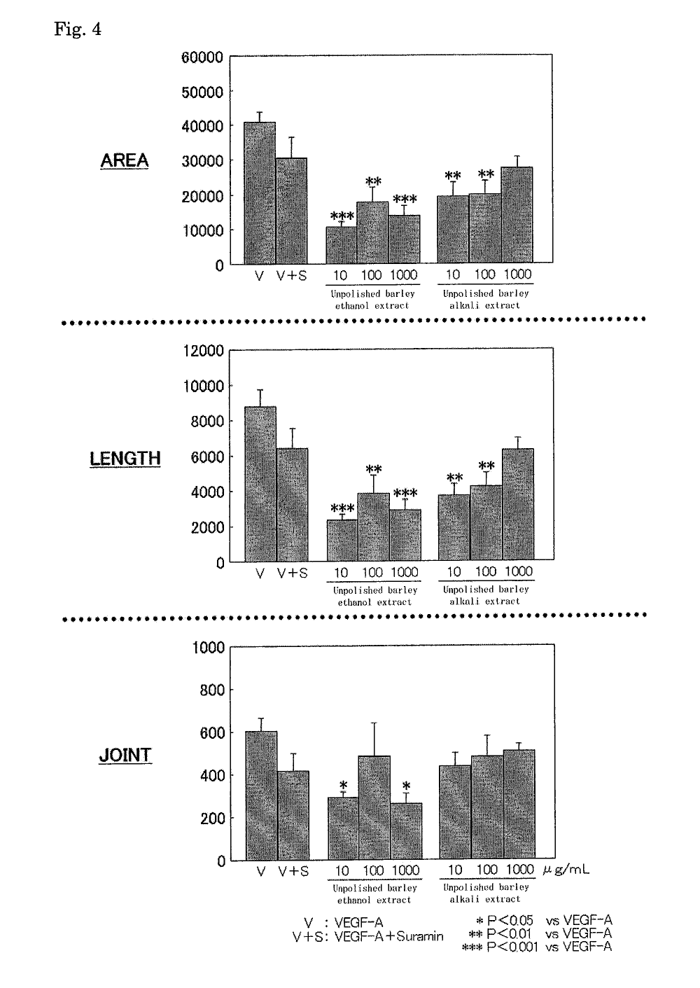

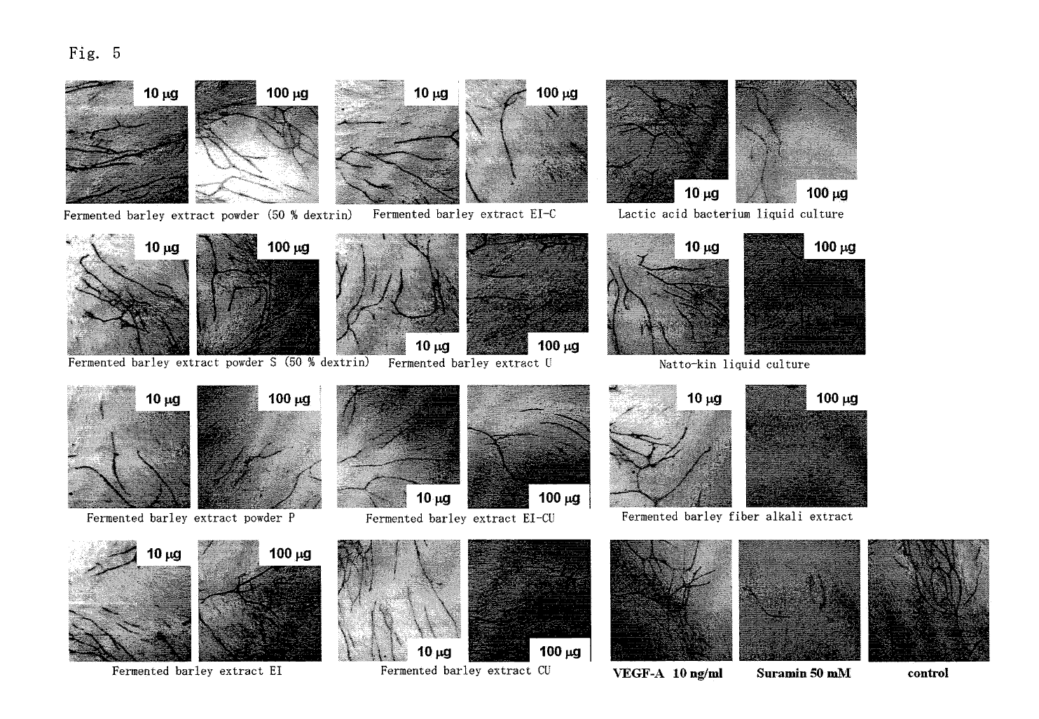

Results: The results are shown in Table 3 (the numerical figure date about the effect on inhibiting vascularization) and FIG. 4.

TABLE 3

| Contents | Samples | | | | AREA | LENGTH | JOINT |
|---|---|---|---|---|---|---|---|
| Negative | VEGF-A alone | | | | 40658 | 8784 | 603 |
| control | | | | | | | |
| Positive | VEGF-A | Suramin | | | 30401 | 6396 | 413 |
| control | | | | | | | |
| Test lot | VEGF-A | Unpolished | 10 | μg | 10509 | 2352 | 291 |
| 1 | VEGF-A | barley | 100 | μg | 17607 | 3800 | 483 |
| | VEGF-A | ethanol | 1,000 | μg | 13673 | 2913 | 261 |
| | | extract | | | | | |
| Test lot | VEGF-A | Unpolished | 10 | μg | 19133 | 371 | 435 |
| 2 | VEGF-A | barley | 100 | μg | 19933 | 4210 | 480 |
| | VEGF-A | alkali | 1,000 | μg | 27578 | 6284 | 504 |
| | | extract | | | | | |

VEGF-A as a vascularization-promoting factor was added at an amount of 5 ng/ml (as the final concentration)
Suramin as a positive control with an observed effect on inhibiting vascularization was added at an amount of 50 μM (as the final concentration)

The results shown in Table 3 and FIG. 4 indicate that the unpolished barley ethanol extract significantly inhibited lumen formation at any of 10 μg/ml, 100 μg/ml and 1000 μg/ml and that the unpolished barley alkali extract completely inhibited lumen formation at 100 μg/ml.

### Example 3

### Experiment 3

Experiment about Vascularization Inhibition and TOF-MS Measurement

### <Names of Samples>

No. 1-1: fermented barley extract powder (50% dextrin)
No. 1-2: fermented barley extract powder S (50% dextrin)
No. 1-3: fermented barley extract powder P
No. 1-4: fermented barley extract EI
No. 1-5: fermented barley extract EI-C
No. 1-6: fermented barley extract U
No. 1-7: fermented barley extract EI-CU
No. 1-8: fermented barley extract CU
No. 1-9: Lactic acid bacterium liquid culture
No. 1-10: Natto-kin liquid culture (75% dextrin)
No. 1-11: fermented barley fiber alkali extract

### <Method for Preparing Samples for Use in the Experiment>

From the respect of other experiments, the samples used in this experiment were originally powders but the powders were re-dissolved for use in the experiment. However, samples in liquid may satisfactorily be used in the experiment. As an excipient, dextrin at a volume equal to that of the extract was added to some of the samples during pulverization, as described below. Herein, the dextrin content is expressed in percentage by weight (w/w) concentration.

[Powder of Residual Solution from the Distillation of Barley Distilled Spirits] (Fermented Barley Extract Powder)

Barley (polished at 70%) was used as a raw material.

Koji Production: After water was absorbed in barley to 40 w/w % for steaming barley for 40 minutes, the resulting barley was left to cool to 40° C., to which seed koji (white koji species) was inoculated at a ratio of 1 kg to 1 ton of barley for allowing the barley to be left at 38° C. and 95% RH for 24 hours and then at 32° C. and 92% RH for 20 hours, so that barley koji was produced.

Production of barley distilled spirits and a residual solution from the distillation of barley distilled spirits: At the primary charging, 3.6 kl of water and 1 kg (on wet basis) of the cultured bacterial cells of distilled spirits yeast as yeast were added to the barley koji (3 tons as barley) produced by the aforementioned method, to prepare a primary moromi, which was then subjected to fermentation for 5 days (fermentation at first stage). At the second charging, then, 11.4 kl of water and the barley koji produced by the method (6 tons as barley) were added to the primary moromi on completion of the fermentation at the first stage, for 11-day fermentation (fermentation at the second stage). The fermentation temperature was 25° C. for both the first charging and the second charging. The secondary moromi on completion of the fermentation at the second stage was subjected to single distillation according to a general method, to obtain 10 kl of barley distilled spirits and 15 kl of a residual solution from the distillation of barley distilled spirits, at a koji yield of 100%. The residual solution from the distillation of barley distilled spirits was centrifuged under a condition of 8,000 rpm for 10 minutes, to obtain 5 L of a liquid fraction of the residual solution from the distillation of barley distilled spirits. Dextrin at an amount equal to the solid content in the liquid was mixed in the liquid fraction, which was then freeze-dried.

[Non-Adsorbed Fraction of the Residual Solution from the Distillation of Barley Distilled Spirits, which is Never Adsorbed on a Synthetic Adsorbent] (Fermented Barley Extract Powder S)

The residual solution from the distillation of barley distilled spirits as obtained in the distillation step of producing barley distilled spirits was centrifuged under a condition of 8,000 rpm for 10 minutes, to obtain a liquid fraction in the residual solution from the distillation of barley distilled spirits. 25 L of the liquid fraction and 10 L of deionized water were passed in this order through a column (of a resin volume of 10 L) packed with a synthetic adsorbent manufactured by Organo, ie. Amberlite XAD-16 for adsorption and separation, to separate a non-adsorbed fraction of a pass-through fraction never adsorbed on the synthetic adsorbent in the column. Dextrin of an amount equal to the solid content in the resulting non-adsorbed fraction was mixed in the fraction, for freeze-drying with a vacuum freeze-dryer, to obtain a freeze-dried product at 2,400 g.

[Adsorbed Fraction of the Residual Solution from the Distillation of Barley Distilled Spirits, which is Adsorbed on a Synthetic Adsorbent] (Fermented Barley Extract Powder P)

The residual solution from the distillation of barley distilled spirits as obtained in the distillation step of producing barley distilled spirits was centrifuged under a condition of 8,000 rpm for 10 minutes, to obtain a liquid fraction in the residual solution from the distillation of barley distilled spirits. 25 L of the liquid fraction was put in contact with the inside of a column (of a resin volume of 10 L) packed with a synthetic adsorbent manufactured by Organo, ie. Amberlite XAD-16, to obtain an adsorbed fraction in adsorption to the column. Subsequently, 10 L of deionized water was put in contact with the inside of the column on which the adsorbed fraction was adsorbed, to obtain an eluate, which was then removed. Then 10 L of an aqueous 1 wt/vol % sodium hydroxide solution and 10 L of deionized water were put in this order in contact with the inside of the column, to separate 20 L of en eluate containing the adsorbed fraction. 20 L of the eluate was put in contact with the inside of a column (of a resin volume of 10 L) packed with a strongly acidic cation exchange resin IR-120B manufactured by Organo. Subsequently, the liquid was freeze-dried.

[Fraction Powders of the Residual Solution from the Distillation of Barley Distilled Spirits, which are Preliminarily Precipitated with Ethanol] (Individual EI Fractions)

### <Fermented Barley Extract EI>

The residual solution from the distillation of barley distilled spirits as obtained in the distillation step of producing barley distilled spirits was centrifuged under a condition of 8,000 rpm for 10 minutes, to obtain 5 L of a liquid fraction in the residual solution from the distillation of barley distilled spirits. The resulting liquid fraction was concentrated with a vacuum evaporator to a Brix level of 25, to which a 3-fold volume of ethanol was added. The resulting mixture was centrifuged under a condition of 8,000 rpm for 10 minutes to separate an ethanol-insoluble fraction, which was then freeze-dried.

### <Fermented Barley Extract EI-C>

The residual solution from the distillation of barley distilled spirits as obtained in the distillation step of producing barley distilled spirits was centrifuged under a condition of 8,000 rpm for 10 minutes, to obtain a liquid fraction in the residual solution from the distillation of barley distilled spirits. 25 L of the liquid fraction and 10 L of deionized water were passed in this order through a column (a resin volume of 10 L) packed with a cation exchange resin manufactured by Organo, i.e. Amberlite 200CT Na for adsorption and separation, to obtain a non-adsorbed fraction of a pass-through fraction with no adsorption to the cation exchange resin in the column. The resulting liquid fraction was adjusted to a Brix level of 60 with a vacuum evaporator, to which a 3-fold volume of ethanol was added. The resulting mixture was centrifuged under a condition of 8,000 rpm for 10 minutes to separate an ethanol-insoluble fraction, which was then freeze-dried.

### <Fermented Barley Extract EI-CU>

The residual solution from the distillation of barley distilled spirits as obtained in the distillation step of producing barley distilled spirits was centrifuged under a condition of 8,000 rpm for 10 minutes, to obtain a liquid fraction in the residual solution from the distillation of barley distilled spirits. The resulting liquid fraction was adjusted to a Brix level of 10. One liter of the liquid fraction adjusted to a Brix level of 10 was passed through a 500-ml column packed with Amberlite 200CT Na (strongly acidic cation exchange resin) manufactured by Organo, to obtain a fraction never adsorbed onto the ion exchange resin. The resulting fraction never adsorbed onto the ion exchange resin was subjected to a concentration process with an ultrafiltration membrane Centramate Omega 10K (for a molecular weight of 10,000 for fractionation) manufactured by Nippon Pall Corporation, to obtain the resulting concentrate. The resulting liquid fraction was concentrated to a Brix level of 40 with a vacuum evaporator, to which ethanol of a 3-fold volume was added. Then, the resulting solution was centrifuged under a condition of 8,000 rpm for 10 minutes, to separate an ethanol-insoluble fraction, which was then freeze-dried.

### <Fermented Barley Extract U>

The residual solution from the distillation of barley distilled spirits as obtained in the distillation step of producing barley distilled spirits was centrifuged under a condition of 8,000 rpm for 10 minutes, to obtain a liquid fraction in the residual solution from the distillation of barley distilled spirits. The resulting liquid fraction was concentrated with an ultrafiltration membrane Centramate Omega 10K (for a molecular weight of 10,000 for fractionation) manufactured by Nippon Pall Corporation, to obtain the resulting concentrate. The resulting liquid fraction was then freeze-dried.

### <Fermented Barley Extract CU>

The residual solution from the distillation of barley distilled spirits as obtained in the distillation step of producing barley distilled spirits was centrifuged under a condition of 8,000 rpm for 10 minutes, to obtain a liquid fraction in the residual solution from the distillation of barley distilled spirits. The resulting liquid fraction was passed through a column packed with Amberlite 200CT Na (strongly acidic cation exchange resin) manufactured by Organo, to obtain a fraction never adsorbed on the ion exchange resin. The resulting fraction never adsorbed on the ion exchange resin was concentrated with an ultrafiltration membrane Centramate Omega 10K (for a molecular weight of 10,000 for fractionation) manufactured by Nippon Pall Corporation, to obtain the resulting concentrate. The resulting liquid fraction was then freeze-dried.

<Lactic Acid Bacterium Liquid Culture in a Culture Medium of the Residual Solution from the Distillation of Barley Distilled Spirits>

The resulting solution from the distillation of barley distilled spirits as obtained at the distillation step in producing barley distilled spirits was centrifuged under a condition of 8,000 rpm for 10 minutes, to obtain a liquid fraction in the residual solution from the distillation of barley distilled spirits. The liquid fraction was diluted with water for the adjustment to a Brix level of 4, to which glucose was added to 3.6% by weight. Using sodium hydroxide, the resulting solution was adjusted to pH 5.5, for sterilization treatment under a condition of 121° C. for 15 minutes, to obtain a culture medium for culturing a lactic acid bacterium.

Preculture of a Lactic Acid Bacterium with Nisin Generation Potency

50 μl of a strain of Lactococcus lactis IO-1 under storage was inoculated on 10 ml of the TGC culture medium, for static culture at 37° C. for 18 hours, to obtain a liquid culture. 10 ml of the liquid culture was inoculated on 100 ml of the CMG culture medium, for shaking culture at 37° C. and 100 rpm for 3 hours, to obtain a lactic acid bacterium liquid preculture.

Primary Culture of the Lactic Acid Bacterium with Nisin Generation Potency

500 ml of the culture medium for culturing the lactic acid bacterium and 25 ml of the liquid preculture of the lactic acid bacterium were charged in a 2 L-jar fermentor, for batch-wise culturing under conditions of an agitation rate of 250 rpm, a culture temperature of 30° C., a culturing time of 24 hours and pH 5.5. The lactic acid bacterium liquid culture was freeze-dried.

[Natto-Kin Liquid Culture in the Residual Solution from the Distillation of Barley Distilled Spirits as the Culture Medium]

The resulting solution from the distillation of barley distilled spirits as obtained at the distillation step in producing barley distilled spirits was centrifuged under a condition of 8,000 rpm for 10 minutes, to obtain a liquid fraction in the residual solution from the distillation of barley distilled spirits. Using sodium hydroxide, the liquid fraction was adjusted to pH 7.0. The resulting adjusted solution was subjected to a sterilization treatment, to obtain a culture medium for culturing Natto-kin.

Preculture of Natto-Kin

10 g of meat extract and 10 g of peptone were dissolved in 1 L of distilled water. Sodium hydroxide was added to the resulting solution, for the adjustment to pH 7.0, for sterilization treatment under a condition of 121° C. for 15 minutes, to obtain a meat extract culture medium. 5 ml of the meat extract culture medium and one platinum loop of the Miyagino strain as a commercially available Natto-kin were charged into a test tube, for agitation and shaking culture at 42° C. for 15 hours, to obtain a liquid preculture of the Natto-kin.

Primary Culture of the Natto-Kin

One liter of the culture medium for culturing Natto-kin and 5 ml of the liquid preculture of the Natto-kin were charged in a 2-L jar fermentor, for culturing under conditions of an aeration volume of 0.2 vvm, an agitation rate of 300 rpm, and a culturing temperature of 42° C. for 14 days. Dextrin of a volume 3-fold the solid content of the liquid culture of the Natto-kin was mixed in the resulting culture, which was then freeze-dried.

### [Fermented Barley Fiber Alkali Extract]

Using one ton of barley purified to 70%, the moromi of barley distilled spirits was produced, for fermentation/aging for about 2 weeks according to a general method, from which alcohol was separated with a single still. 1.5 kL of a residual solution of the distillation of barley distilled spirits was obtained. One kilo-liter of the resulting residual solution from the distillation of barley distilled spirits was separated as solid and liquid, using a screw press. From the resulting solid, about 50 kg of barley groove was obtained. After the resulting barley groove was dried with a drum dryer, the groove was ground with a roll mill, to obtain 5.5 kg of a powder of barley groove (composition). 50 g of the powder of barley groove was suspended in 1 L of an aqueous 2% Ca(OH)₂ solution, for agitation at ordinary temperature for 6 hours and adjustment to pH 7.0 with HCl, followed by filtration with a filter paper. The filtrate was freeze-dried.

### [Preparation of Experimental Samples, Experimental Method and Results]

### 1. Experimental Samples

### 1-1. Sample Preparation

### 1-1-1. For Use in Vascularization Inhibition Experiments

Each of the samples Nos. 1-1 through 1-11 was weighed at 1,000 μg, dissolved in 1 ml of a culture medium and sterilized by filtration (0.22 μm). The resulting filtrate was diluted 10-fold (two times) to prepare samples at concentrations of 10 μg/ml to 100 μg/ml.

### 1-1-2. For Use in TOF-MS Measurement Tests

After the samples Nos. 1-1 through 1-4 were dissolved in water to 10 mg/ml, the samples were diluted to concentrations of 1 mg/ml, 100 μg/ml, 10 μg/ml, 1 μg/ml and 0.1 μg/ml.

The sample No. 1-11 was poorly dissolved.

### 2. Experimental Method and Results

### 2-1. Experiment for Vascularization Inhibition

Human vascular endothelial cells and fibroblasts were co-cultured at their optimal concentrations. At the proliferation state at an early stage of luminal formation, the individual samples (1-1-1) were added for 11-day culturing (the culture media containing the samples were exchanged 4, 7 and 9 days later). Subsequently, luminal formation was observed with a microscope by staining the formed lumen-like network structure with a mouse anti-human CD31/goat anti-mouse IgG AlkP conjugate. So as to evaluate the effect on inhibiting vascularization, the formed lumen-like network structure was observed. The results are shown in FIG. 1. As apparently shown in FIG. 1, the fermented barley fiber alkali extract and the fermented barley extract P significantly inhibited lumen formation.

### 2-2. TOF-MS Measurement Tests

### 2-2-1. Conditions for the Measurement

Under both the positive ion- and negative ion conditions, the measurement and detection were done by a reflector mode.

Beta-cyclodextrin (M.W. 1135.12) was used as a standard substance for the measurement, while the dimer of DNBA (positive ion mode 273 and negative ion mode 307) and beta-cyclodextrin were used for calibration.

### 2-2-2. Sample Preparation

As the matrix, aqueous 10 mg/ml 2,5-dihydroxybenzoic acid (DHBA) solution or an aqueous 20% ethanol solution was used. A sample solution at each concentration was mixed with the matrix solution at a ratio of 1:1 or 2:2. Each mixture solution of 1 μl was applied onto a plate for the measurement, which was then spontaneously dried for the measurement by the negative ion mode.

By the measurement by the positive ion mode, main peaks of polysaccharides are generally observed at [M+Na]+ or [M+K] as the main peaks. With reference to the report of the MALDI-TOF MS measurement of a cyckodextrin derivative [B. Chankvetadze et al., Carbohydrate Research, 287, 139-555 (1996)], therefore, a mixture solution of a sample solution: the matrix solution: aqueous 30 mM NaCl solution at 1:1:1 was also prepared, for use in the measurement by the positive ion mode.

Consequently, an identical pattern was observed for the four samples (the pattern is not shown).

By the positive ion mode, peaks from a peak around 650 Da to a peak around about 3000 Da were observed at an interval of about 210 Da, while the peak intensities were lowered following the increase of the molecular weight. By the negative ion mode, the same performance was observed from a peak at about 580 Da at an interval of about 210 Da.

Like the peak at 645 Da by the positive ion mode and the peak at 576 Da by the negative ion mode, an interval between the peaks of close values by the positive/negative ion modes was about 69 Da. 69 Da corresponds to about 3Na+, which may be due to the addition of aqueous NaCl solution during the sample preparation.

About a possibility of peaks observed at an interval of 210 Da, the following two reasons may be suggested.

1) A modified sugar moiety corresponding to 210 Da is cleaved via laser irradiation.
2) A mixture of homo-polysaccharides composed of a sugar corresponding to 210 Da.

The possibility about the reason 1) was examined. As far as the examination was concerned, however, not any modification of a sugar corresponding to 210 Da was found. Regarding the possibility about the reason 2), the molecular weight of a sugar with carboxyl group (—COOH) in the modified moiety was 209 Da, which was a very close value.

### [Reference]

### -NMR Measurement

So as to examine the possibility of the reason 2), 1H-NMR of the sample 2-1 at 7 mg/ml in deuterium was measured (the chart is not shown). The resulting chart was compared with the spectrum of beta-cyclodextrin, indicating a very complicated signal at 3 to 5 ppm in addition to a signal at 1 to 3 ppm, which may be derived from a sugar. Additionally, a signal was observed at 6.5 to 7.5 ppm, indicating that amide proton or a compound with an aromatic ring might be contained. Thus, it is hard to elucidate the structure of polysaccharides.

The results obtained at this time indicate that samples may contain polysaccharides of about 600 to about 3000 Da and low-molecular compounds. Because a possibility of the existence of a compound of 3000 Da or more still remains, conditions for the measurement would be examined to continue the measurement.

### Example 4

### Experiment 4

### Experiment for Vascularization Inhibition

### <Names of Samples>

The names of samples are shown in Table 4.

A1, A2: Negative control
A3, A4: Positive control
D1 through D3: Powder of residual solution from the distillation of barley distilled spirits, which is adsorbed with a synthetic adsorbent (Fermented Barley extract powder P)<
<Method for Preparing Samples for Use in Vascularization inhibition>

From the respect of other experiments, the samples used in this experiment were originally powders but the powders were re-dissolved for use in the experiment. However, samples in liquid may satisfactorily be used in the experiment. As an excipient, dextrin at a volume equal to that of the extract was added to some of the samples during pulverization, as described below. Herein, the dextrin content is expressed in percentage by weight (w/w) concentration.

[Powder of a Residual Solution from the Distillation of Barley Distilled Spirits, Which is Adsorbed with Synthetic Adsorbent]

### (Fermented Barley Extract Powder P)

The sample described in the column 0062 was used.

### [Preparation of Experimental Specimen, Experimental Method and Results]

### 1. Experimental Specimen

### 1-1. Sample Preparation

### 1-1-1. For Experiment for Inhibiting Vascularization

1,000 μg of each specimen was weighed and dissolved in 1 ml of a culture medium. The resulting culture medium was sterilized by filtration (0.22 μm), diluted 10 fold (three times), to prepare samples at concentrations of 10 μg/ml to 1,000 μg/ml as shown in Table 4 (the contents of test samples for assessing vascularization inhibitory effect). Similarly, the negative control samples and the positive control samples as shown in Table 4 were prepared.

### 2. Experimental Method and Results

2-1. Experiment about Vascularization Inhibition

Human vascular endothelial cells and fibroblasts were co-cultured at their optimal concentrations. At the proliferation state at an early stage of luminal formation, the individual samples (1-1-1, the negative controls, the positive controls) were added for 11-day culturing (the culture media containing the samples were exchanged 4, 7 and 9 days later). Subsequently, luminal formation was observed with a microscope by staining the formed lumen-like network structure with a mouse anti-human CD31/goat anti-mouse IgG AlkP conjugate. Regarding the effect on inhibiting vascularization, the formed lumen-like network structure was evaluated.

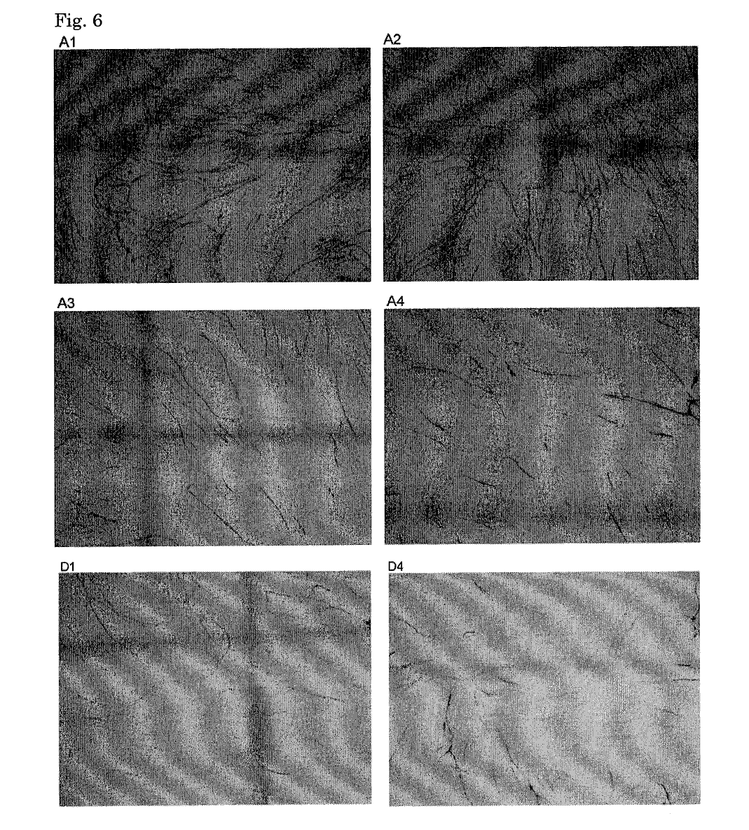

The results are shown in Table 5 and FIG. 6. Photographs of blood tube tissues were incorporated as digital data, to measure the area of the blood tube region (black part) at several positions randomly selected, to determine the inhibitory effect of vascularization. Specifically, a smaller numerical figure of AREA in Table 5 shows a sample with a higher vascularization inhibitory effect.

TABLE 4

| No. | Contents | Samples | |
|---|---|---|---|
| A1 | Negative control | VEGF-A alone | |
| A2 | | VEGF-A alone | |
| A3 | Positive control | VEGF-A | suramin |
| A4 | | VEGF-A | suramin |
| D1 | Test lot 1 | VEGF-A | Fermented barley extract P |
| | | | 10 μg |
| D2 | | VEGF-A | Fermented barley extract P |
| | | | 100 μg |
| D3 | | VEGF-A | Fermented barley extract P |
| | | | 1,000 μg |

| | |
|---|---|
| D4 | Culture medium alone |

VEGF-A as a vascularization-promoting factor was added at an amount of 5 ng/ml (as the final concentration)
Suramin as a positive control with an observed effect on inhibiting vascularization was added at an amount of 25 μM (as the final concentration)

TABLE 5

| Sample | Microscopic | | | | |
|---|---|---|---|---|---|
| No. | photograph | AREA | LENGTH | JOINT | PATH |
| A1 | A1 in FIG. 6 | 76855 | 11872 | 69 | 267 |
| A2 | A2 in FIG. 6 | 92911 | 14727 | 96 | 351 |
| A3 | A3 in FIG. 6 | 27940 | 4583 | 11 | 107 |
| A4 | A4 in FIG. 6 | 33301 | 5311 | 16 | 128 |
| D1 | D1 in FIG. 6 | 32842 | 5346 | 22 | 130 |
| D2 | D2 in FIG. 6 | 35218 | 6119 | 18 | 132 |
| D4 | D4 in FIG. 6 | 30479 | 5510 | 22 | 135 |

An apparent effect on inhibiting vascularization was observed in the fermented barley extract powder P.

### Example 5

### Experiment 5

### Experiment for Vascularization Inhibition

### <Names of Samples>

The names of samples are shown in Table 6.

### Method for Preparing Samples (1) Through (9) for Use in Vascularization Inhibition

From the respect of other experiments, the samples used in this experiment were originally powders but the powders were re-dissolved for use in the experiment. However, samples in liquid may satisfactorily be used in the experiment. As an excipient, dextrin at a volume equal to that of the extract was added to some of the samples during pulverization, as described below. Herein, the dextrin content is expressed in percentage by weight (w/w) concentration.

[Powder of Residual Solution from the Distillation of Barley Distilled Spirits, which is Adsorbed onto a Synthetic Adsorbent] (Fermented Barley Extract Powder P)

See the column 0062.

### [Preparation of Experimental Specimen, Experimental Method and Results]

### 1. Experimental Specimen

### 1-1. Sample Preparation

### 1-1-1. For Experiment for Inhibiting Vascularization

1,000 μg of each specimen was weighed and dissolved in 1 ml of a culture medium. The resulting culture medium was sterilized by filtration (0.22 μm) and diluted 10 fold (three times), to prepare samples at concentrations of 10 μg/ml to 1,000 μg/ml.

### 2. Experimental Method and Results

2-1. Experiment about Vascularization Inhibition

Human vascular endothelial cells and fibroblasts were co-cultured at their optimal concentrations. At the proliferation state at an early stage of luminal formation, the individual samples were added for 11-day culturing (the culture media containing the samples were exchanged 4, 7 and 9 days later). Subsequently, luminal formation was observed with a microscope by staining the formed lumen-like network structure with a mouse anti-human CD31/goat anti-mouse IgG AlkP conjugate. As standards for the evaluation, VEGF-A promoting luminal formation was added at 10 ng/ml, while suramin inhibiting luminal formation was added at 50 μM, in the same manner. A control was prepared without any addition. Regarding the effect on inhibiting vascularization, the formed lumen-like network structure was evaluated.

Method: A kit manufactured by KURABO was used for measurement. The lumen formation state (the area, the length and the number of branches) was observed at four points. VEGF as a vascularization promoting factor and suramin as an agent for suppressing vascularization were concurrently present in a control, so as to compare the lumen formation state in the co-existence of VEGF and each sample.

### (Comparison in Activity of Inhibiting Vascularization Between Suramin and Sample)

Concentration of a sample added: 10, 100 and 1000 μg/ml (Suramin was added in all of the cases at the same concentration; 50 μM)

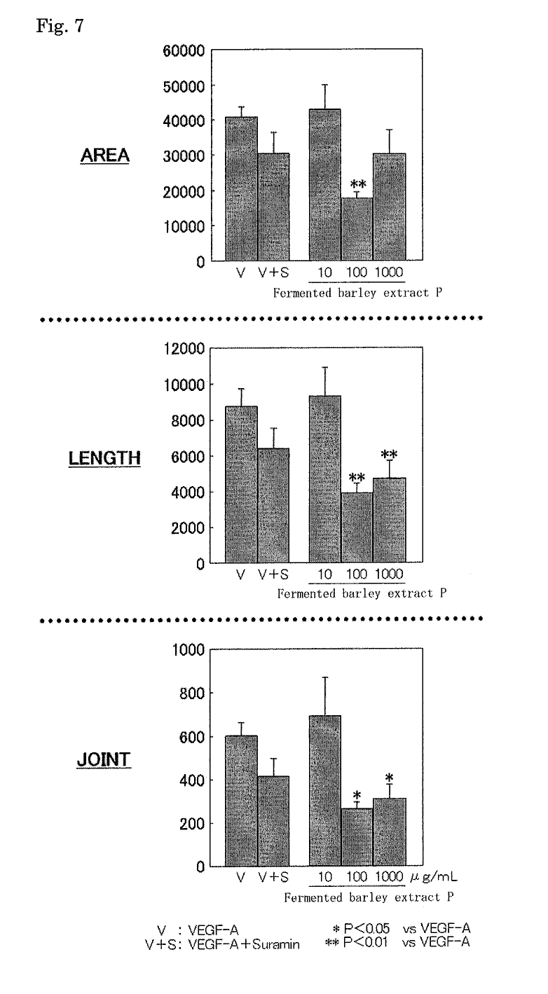

Results: The results are shown in Table 6 (numerical figure date about the effect on inhibiting vascularization) and FIG. 7.

TABLE 6

| Contents | Samples | | | | AREA | LENGTH | JOINT |
|---|---|---|---|---|---|---|---|
| Negative | VEGF-A alone | | | | 40658 | 8784 | 603 |
| control | | | | | | | |
| Positive | VEGF-A | Suramin | | | 30401 | 6396 | 413 |
| control | | | | | | | |
| Test lot | VEGF-A | Fermented | 10 | μg | 42986 | 9338 | 694 |
| 1 | VEGF-A | barley | 100 | μg | 17710 | 3916 | 265 |
| | VEGF-A | extract P | 1,000 | μg | 30175 | 4714 | 310 |

VEGF-A as a vascularization-promoting factor was added at an amount of 5 ng/ml (as the final concentration)
Suramin as a positive control with an observed effect on inhibiting vascularization was added at an amount of 50 μM (as the final concentration)

The results shown in Table 6 and FIG. 7 indicate that 100 μg of the fermented barley extract P completely inhibited lumen formation.

### Example 6

Assessment of Vascularization-Inhibiting Action In Vivo of Powder of Residual Solution of the Distillation of Barley Distilled Spirits (Fermented Barley Extract), Powder of Residual Solution of the Distillation of Barley Distilled Spirits, which is Adsorbed onto a Synthetic Adsorbent (Fermented Barley Extract P), and Unpolished Barley Ethanol Extract Fraction in a Vascularization Model in Rats with Sponge Subcutaneously Grafted

### [Experimental Material and Method]

1. As the fermented barley extract powder, the fermented barley extract powder P and the unpolished barley ethanol extract fraction powder, the samples described in the columns 0060, 0062 and 0044 were used.

### 2. Feed Composition

Each testing substance was mixed into a feed (AIN-76 manufactured by Oriental Yeast Co., Ltd.) to 1% (in case of the fermented barley extract powder, the fermented barley extract powder P, and the unpolished barley ethanol extract fraction powder) or to 5% (in case of the fermented barley extract powder), for use in the experiments. Herein, the standard feed was given to the control group. The compositions of the feeds are shown in Table 7.

TABLE 7

| | | Fermented | Fermented | Fermented | Unpolished barley |
|---|---|---|---|---|---|
| | | barley | barley | barley | ethanol extract |
| Raw material | control | extract at 1% | extract at 5% | extract P at 1% | fraction at 1% |
| Casein | 20 | 20 | 20 | 20 | 20 |
| DL-Methionine | 0.3 | 0.3 | 0.3 | 0.3 | 0.3 |
| Corn starch | 15 | 15 | 15 | 15 | 15 |
| Sucrose | 45 | 44 | 40 | 44 | 44 |
| Corn oil | 5 | 5 | 5 | 5 | 5 |
| Mineral mixture a) | 3.5 | 3.5 | 3.5 | 3.5 | 3.5 |
| Vitamin mixture a) | 1 | 1 | 1 | 1 | 1 |
| Choline bitartrate | 0.2 | 0.2 | 0.2 | 0.2 | 0.2 |
| Cellulose powder | 5 | 5 | 5 | 5 | 5 |
| Fermented barley | — | 6 | 10 | — | — |
| extract powder | | | | | |
| Fermented barley | — | — | — | 6 | — |
| extract powder P | | | | | |
| Unpolished barley | — | — | — | — | 6 |
| ethanol extract | | | | | |
| fraction powder | | | | | |
| Dextrin | 5 | — | — | — | — |
| Total | 100 | 100 | 100 | 100 | 100 |

Numerical figure: in %
A: AIN-76 (Nutrition 110(8) in 1980)
Containing dextrin at 5% in all the tested substances

### 3. Animals Used

Male Crlj:CD (SD) rats of 7 weeks old were purchased (Nippon Charles River), for one-week quarantine and feeding. At the 8 weeks old, then, the animals were used for this experiment. The rats were individually fed in one lot of a 5-series metal net cage. The rats were then fed in an animal feeding chamber adjusted to environment at a temperature of 23±2°, a humidity of 30 to 80%, a ventilation number of 12 times or more/hour, and an illumination period of 12 hours (in the lighting period of 6:30 to 18:30). The feeds and running water were fed at libitum.

### 4. Experimental Method

After the completion of the quarantine and feeding period, the rats were grouped into 8 rats per one group so as to make the mean body weight in each group uniform. It was started to give the mixture feeds. Two weeks after the start of feeding, a sponge (of a diameter of 13 mm and a thickness of 5 mm) was implanted in the back skin of the rats under ether anesthesia. Subsequently, the mixture feeds were continuously given for another 2 weeks. After the completion of the feeding period, the rats were subjected to euthanasia via whole blood drawing under ether anesthesia, from which a granulated tissue including the sponge was resected and weighed. A part of the resected granulated tissue was homogenized and centrifuged. The hemoglobin concentration in the resulting supernatant was measured with a hemoglobin assay kit (Hemoglobin B Test Wako, Wako Pure Chemical Co., Ltd.). According to a routine method, the vascular endothelial cells in the resected granulated tissue were stained immunologically with CD34 to calculate the blood tube area ratio. Additionally, histopathological images of typical examples in each group were photographed. Once a week after the start of the test, body weight and feed intake were measured.

### 5. Statistical Treatment

The measured values were expressed as mean value±standard deviation.

Significance from the control group was determined by the Student's t-test at the significance level of 5% or less.

### [Results]

### 1. Body Weight and Feed Intake

No significant difference in body weight or feed intake was observed among the individual rat groups.

### 2. Weight of Granulated Tissues (Sponge)

FIG. 8 shows the mean value and standard deviation of the weight of granulated tissues (sponge) in the individual rat groups. Compared with the control group, the weight of granulated tissues (sponge) in the group fed with the fermented barley extract powder at 5% and in the group fed with the fermented barley extract powder P at 1% was significantly low. In the group fed with the unpolished barley ethanol extract fraction powder at 1%, the weight thereof showed a tendency of small values.

### 3. Hemoglobin Concentration in Granulated Tissues

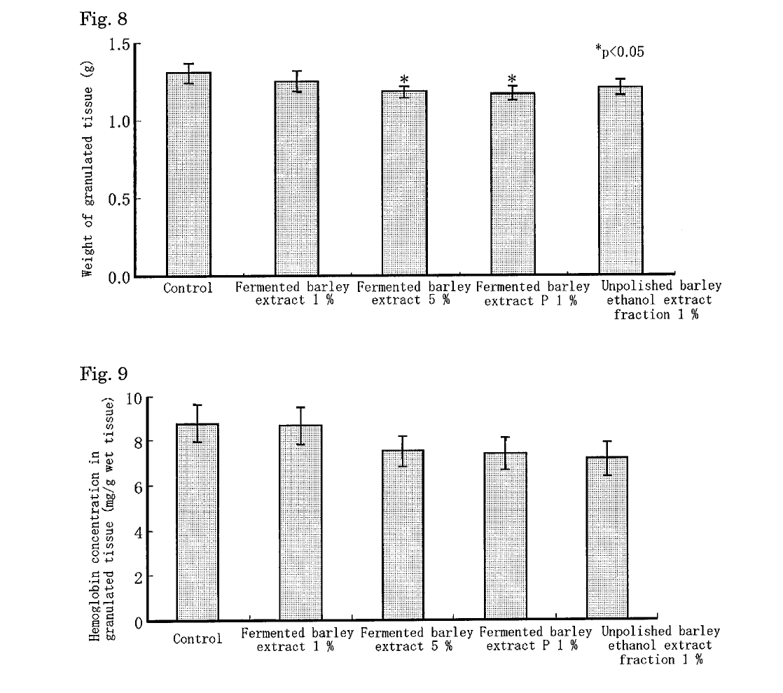

FIG. 9 shows the mean value and standard deviation of the hemoglobin concentration of granulated tissues in the individual rat groups. Compared with the control group, the hemoglobin concentration of granulated tissues in the group fed with the fermented barley extract powder at 5%, in the group fed with the fermented barley extract powder P at 1% and in the group fed with the unpolished barley ethanol extract fraction powder at 1% was not significantly different. The hemoglobin concentrations in these groups were more or less at small values.

### 4. Blood Tube Area Ratio in Granulated Tissues

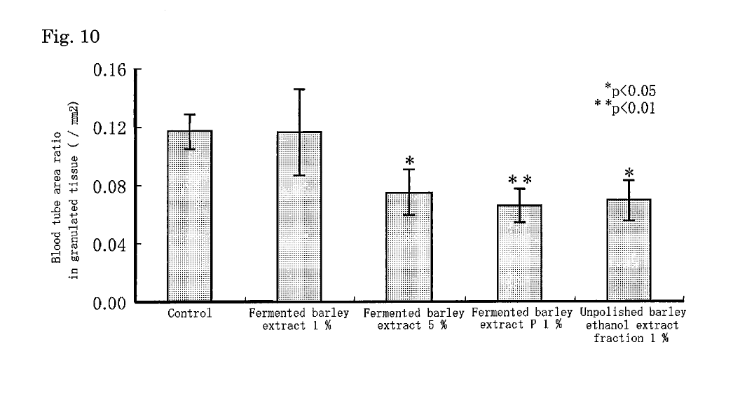

FIG. 10 shows the mean value and standard deviation of the vascularization inner cavity area ratio obtained by CD34 immunostaining of endothelial cells in granulated blood tube in each rat group. FIG. 11 shows histopathological images in typical examples in the individual groups. Compared with the control group, the blood tube area ratio in the group fed with the fermented barley extract powder at 5%, in the group fed with the fermented barley extract powder P at 1% and in the group fed with the unpolished barley ethanol extract fraction powder at 1%, was significantly small.

The aforementioned results indicate that all of the fermented barley extract powder, the fermented barley extract powder P and the unpolished barley ethanol extract fraction powder had the action of inhibiting vascularization in granulated tissues.

### INDUSTRIAL APPLICABILITY

The composition of the invention comprises the application of the vascularization inhibition of the active ingredient derived from barley as a plant of the family Gramineae, which has been essential to humans since prehistoric times and has been familiar as a very healthy food as described in for example traditional medical textbooks in Japan and also derived from fermented barley. The composition of the invention is highly applicable as a functional food material.

1-15. (canceled)

16. Use for inhibiting vascularization of one or more compositions selected from the group consisting of a husked but unpurified barley ethyl alcohol extract fraction, a husked but unpurified barley alkali extract fraction, a non-adsorbed fraction of a residual solution from the distillation of barley distilled spirits, which is not adsorbed with a synthetic adsorbent, an adsorbed fraction of a residual solution from the distillation of barley distilled spirits, which is adsorbed with a synthetic adsorbent, a fraction of a residual solution from the distillation of barley distilled spirits, which is precipitated with ethanol, and a lactic acid bacterium culture obtained by culturing a lactic acid bacterium in a culture medium containing a residual solution from the distillation of barley distilled spirits.

17. Use for inhibiting vascularization according to claim 16, for therapeutically treating or preventing a disease for which vascularization should be inhibited.

18. Use for inhibiting vascularization according to claim 17, where the disease for which vascularization should be inhibited is a disease with an etiology of abnormal vascularization in tumor or cancer, or chronic inflammation or retinopathy.

19. Use for inhibiting vascularization according to claim 16 or 17, where the composition is in a form selected from the group consisting of food additives, food materials, foods and drinks, pharmaceutical products under regulations by the Ministry of Health and Labor in Japan and quasi-pharmaceutical products under regulations by the Ministry of Health and Labor in Japan, and feeds for inhibiting vascularization.

20. Use for inhibiting vascularization according to claim 19, where the foods and drinks are functional foods, nutritional supplement foods or healthy foods and drinks for inhibiting vascularization.

21. Use for inhibiting vascularization according to claim 19, where the feeds are feeds for cattle, poultry and pets for inhibiting vascularization.

22. A method for preventing and/or therapeutically treating a disease for which vascularization should be inhibited, comprising a step of administering one or more compositions selected from the group consisting of a husked but unpurified barley ethyl alcohol extract fraction, a husked but unpurified barley alkali extract fraction, a non-adsorbed fraction of a residual solution from the distillation of barley distilled spirits, which is not adsorbed with a synthetic adsorbent, an adsorbed fraction of a residual solution from the distillation of barley distilled spirits, which is adsorbed with a synthetic adsorbent, a fraction of a residual solution from the distillation of barley distilled spirits, which is precipitated with ethanol, and a lactic acid bacterium culture obtained by culturing a lactic acid bacterium in a culture medium containing a residual solution from the distillation of barley distilled spirits, to a patient afflicted with a disease for which vascularization should be inhibited.

23. A method for preventing and/or therapeutically treating a disease for which vascularization should be inhibited according to claim 22, where the disease for which vascularization should be inhibited is a disease with an etiology of abnormal vascularization in tumor or cancer, or chronic inflammation or retinopathy.
